(19) United States
(12) Patent Application Publication
(10) Pub. No.: US 2026/0191482 A1
(43) Pub. Date: Jul. 9, 2026

(54) Chair Apparatus For Breast Imaging

(71) Applicant: United States Government As Represented By The Department Of Veterans Affairs, Washington, DC (US)

(72) Inventors: Natalie Mandel, Cleveland, OH (US); Frank P. Zitko, Cleveland, OH (US); Raychel M. Testa, Cleveland, OH (US)

(21) Appl. No.: 19/010,729

(22) Filed: Jan. 6, 2025

Related U.S. Application Data
(60) Provisional application No. 63/618,042, filed on Jan. 5, 2024.

(51) Int. Cl. A61B 6/04; A61B 6/50

(52) U.S. Cl. CPC A61B 6/0478; A61B 6/502

(57) ABSTRACT

A chair apparatus for supporting a patient adjacent an imaging unit including a generator assembly and an image receptor plate is disclosed. The chair apparatus has a base assembly and a seat assembly supported by the base assembly. The seat assembly includes the seat portion having a body with a front end and a rear end. The front end of the body of the seat portion defines a recess therein. The recess extends rearwardly into the body toward the rear end of the body and is configured to slidably receive a portion of the generator assembly therein. Also disclosed is a seat assembly for a chair apparatus.

### CROSS-REFERENCE TO RELATED APPLICATIONS

This application claims priority to and the benefit of U.S. Provisional Patent Application No. 63/618,042, filed Jan. 5, 2024, the entirety of which is hereby incorporated by reference herein.

### FIELD

This disclosure is generally related to medical devices and, more particularly, to chair apparatuses and methods of use thereof in combination with radiological or imaging systems when performing breast imaging and related operations.

### BACKGROUND

A needle localization is a breast biopsy in which a guide wire is placed into the breast of a patient to identify a precise location of abnormal breast tissue. Some biopsies require a patient to stand while the wire is being placed so that the x-ray tube of the imaging unit may be positioned beneath the breast of the patient. Positioning the x-ray tube of the imaging unit below the patient's breast typically requires that the patient stand for prolonged periods of time as the x-ray tube is rotated 180° from its typical position above the patient's breast. This approach, referred to as “from below,” can be difficult for the patient as the patient must remain still while standing during the procedure. Not only is the patient potentially fatigued, but the safety of the procedure is possibly compromised as the positioning of the patient can be awkward for the performing physician and technicians as well. Note, similar issues may exist not only when capturing x-ray images, but also ultrasound images, magnetic resonance images, etc.

During a typical needle localization, mammogram or other breast imaging procedure, the patient may be seated in most cases while the x-ray tube is disposed above the breast of the patient and the corresponding image tray is disposed beneath. Although chairs are available for use during some breast procedures, they do not allow the flexibility of inverting the x-ray tube to the position beneath the patient's breast. Standard chairs do not allow this because the x-ray tube typically abuts the front-end of the chair which does not allow the patient to get close enough to the imaging unit for proper imaging and/or needle localization.

From the foregoing, one recognizes the need for systems and methods that may be utilized to facilitate the efficiency and comfort of both patients and technicians during breast imaging and biopsy procedures.

### SUMMARY

Embodiments of the disclosed chair apparatus are for supporting a patient adjacent an imaging unit having a generator assembly and an image receptor plate. The embodiments of the chair apparatus facilitate performing breast imaging, mammograms and breast biopsies by allowing the patients to remain in a seated position when performing the procedures “from below.” Embodiments of the disclosed chair apparatus may include a base assembly configured to allow the chair apparatus to be moved along a support surface disposed beneath the chair apparatus, and a seat assembly supported by the base assembly. Preferably, the seat assembly includes a seat portion having a body, the body having a front end and a rear end, wherein the front end of the body defines a recess therein. The recess extends rearwardly into the body of the seat portion toward the rear end of the body and is configured to slidably receive a portion of the generator assembly therein during “from below” procedures when the generator assembly is inverted.

Also disclosed herein is a seat portion for a chair apparatus for supporting a patient adjacent an imaging unit including a generator assembly and an image receptor plate, the chair apparatus having a base assembly configured to allow the chair apparatus to be moved along a support surface disposed beneath the chair apparatus and a seat assembly supported by the base assembly, the seat assembly including the seat portion. The seat portion includes a body having a front end and a rear end. The front end of the body of the seat portion defines a recess therein, the recess extending rearwardly into the body toward the rear end of the body. The recess defined by the front end of the body is configured to slidably receive a portion of the generator assembly therein.

Yet other embodiments of the present invention provide an imaging system for performing an exam, such as a breast examination, on a patient wherein the patient may remain seated during “from below” procedures. The imaging system may include an imaging unit having a generator assembly and an image receptor plate, the chair apparatus having a base assembly configured to allow the chair apparatus to be moved along a support surface, and a seat assembly supported by the base assembly. Preferably, the seat assembly includes a seat portion having a front end, a rear end, and a left side and a right side extending therebetween, wherein the front end of the seat portion defines a recess therein. The recess extends rearwardly into the seat portion toward the rear end of the seat portion, and the recess is configured to slidably receive a portion of the generator assembly therein.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1A and 1B are perspective views of an imaging system having a chair apparatus and a breast imaging unit in accordance with an embodiment of the present disclosure. FIG. 1A shows the chair apparatus spaced from the breast imaging unit, and FIG. 1B shows the chair apparatus receiving a generator assembly of the breast imaging unit within a recess in a seat portion of the chair apparatus.

FIG. 2 is a front view of the chair apparatus shown in FIGS. 1A and 1B.

FIGS. 3A and 3B are perspective views of the seat portion of the chair apparatus shown in FIGS. 1A-1C.

FIGS. 4A through 4D are perspective views of a seat portion for the chair apparatus of FIGS. 1A-1C in different configurations.

FIGS. 5A and 5B are perspective views of a seat portion for the chair apparatus of FIGS. 1A-1C in different configurations. FIG. 5C is a side view of the seat portion of FIG. 5B. FIG. 5D is a lower rear perspective view of the seat portion for the chair apparatus of FIGS. 1A-1C, with a pair of flaps in a raised configuration and a third flap in a lowered configuration.

FIGS. 6A through 6C are perspective views of a seat portion for the chair apparatus of FIGS. 1A-1C in different configurations.

FIG. 7A is an underside perspective view of certain elements of an exemplary seat portion including a third flap in a first position. FIG. 7B is an underside perspective view of the elements of FIG. 7A, with the third flap in a second position.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” can optionally include plural referents unless the context clearly dictates otherwise. For example, unless the context dictates otherwise, use of the term “a pad” can represent disclosure of embodiments in which only a single such pad is provided, as well as embodiments in which a plurality of such pads are provided, and so forth.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, can, in alternative embodiments, also include any combination of members of that list.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

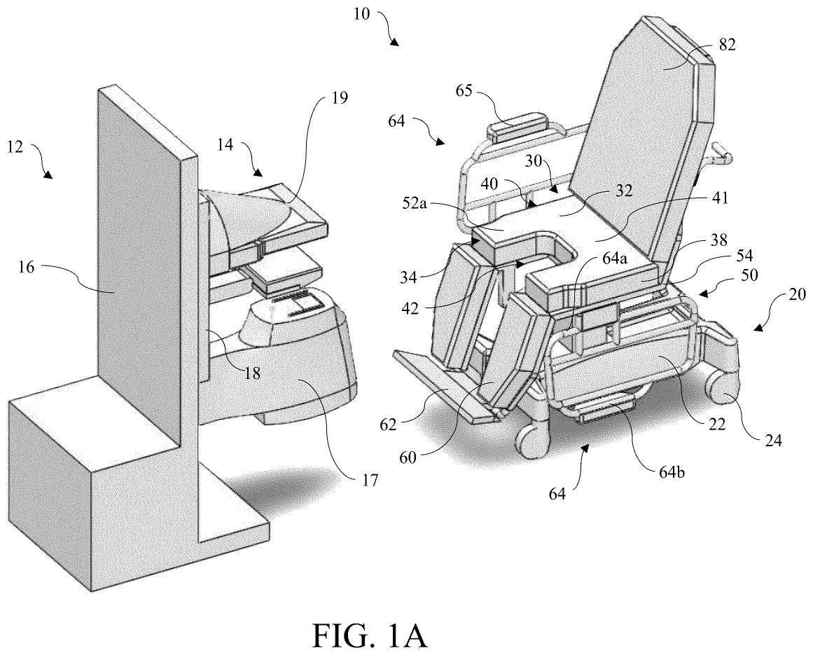

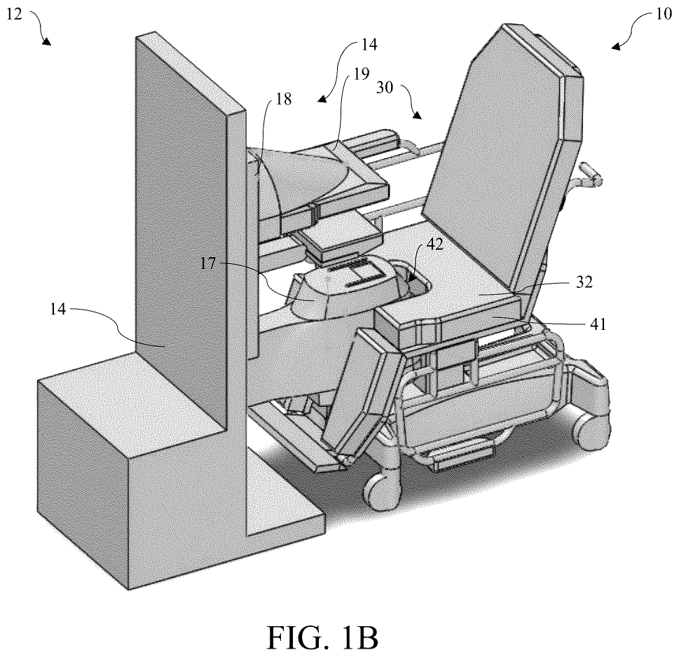

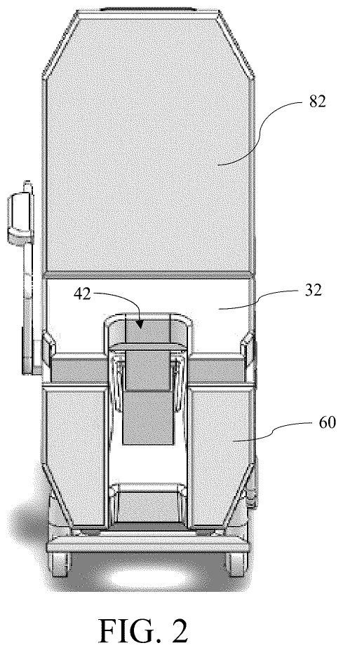

Referring now to the figures, an exemplary embodiment of a chair apparatus 10 for use in breast imaging and biopsy procedures is shown. Such breast imaging and biopsy procedures include, for example and without limitation, 2D mammography, 3D mammography (e.g., tomosynthesis), thermography, and/or ultrasound. As seen in FIGS. 1A and 1B, the chair apparatus 10 can comprise a base assembly 20, a seat assembly 30, and a vertical lift assembly 50 that adjustably connects the seat assembly 30 to the base assembly 20. The base assembly 20 can have a platform 22 and a plurality of castors 24 so that the chair apparatus 10 may be easily maneuvered along a support surface disposed therebeneath. The vertical lift assembly 50 can be a telescoping arm that may be used to adjust the height of the seat assembly 30 with respect to the base assembly 20. The vertical lift assembly 50 may be manually adjusted prior to a patient being received on the seat assembly 30. However, in various embodiments, the vertical lift assembly 50 may be pneumatically or hydraulically assisted so that the height of the seat assembly 30 is adjustable with respect to the base assembly 20 while the patient is supported on the seat assembly 30. Adjusting the height of the seat assembly 30 and, therefore, the patient may be required when positioning the patient with respect to the corresponding imaging system 12, as shown in FIGS. 1A and 1B, and discussed in greater detail below.

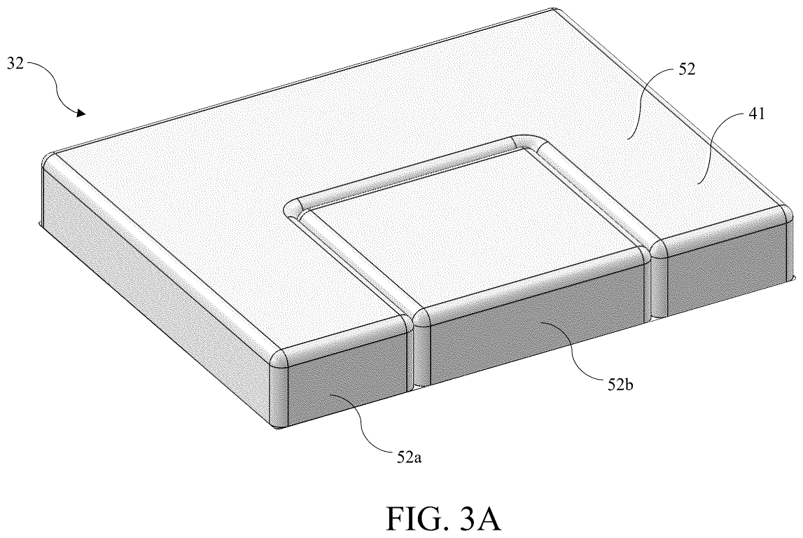

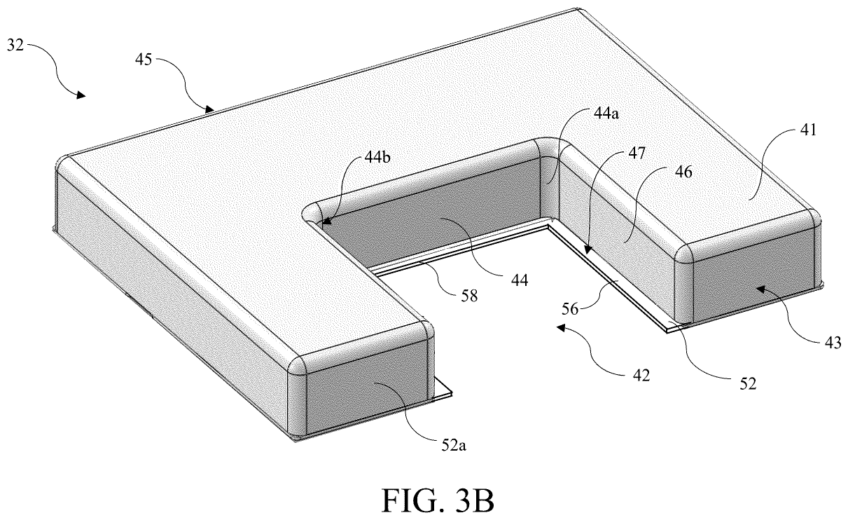

Referring to FIGS. 1A and 3A-3B, the seat assembly 30 can comprise a seat portion 32 and a back rest 82. In additional aspects, the seat assembly 30 can comprise a pair of leg supports 60 at least one arm rest 64 (e.g., a pair of arm rests 64). Each arm rest 64 can be disposed on a corresponding left side 38 or right side 40 of the seat portion 32, and includes a proximal end 64a that is pivotably secured to a frame 54 of the seat portion 32 and a distal end 64b that receives a cushion 65 thereon. As shown, one or both arm rests 64 can be pivotable between a first upright position (see the right-side arm rest 64 in FIG. 1A) in which the distal end 64b of the arm rest 64 is disposed above the seat portion 32, and a second stowed position in which the entire arm rest 64 is disposed below the seat portion 32. When disposed in the upright position, the patient may rest his forearms on the cushions 65 of the armrests 64. The stowed position, that being shown by the left arm rest 64 in FIG. 1A, facilitates patient entry into the chair apparatus 10. Additionally, the stowed position facilitates access to the patient by the attending physician or technician from the left side of the chair apparatus 10. As noted, both armrests 64 may be placed in the stowed position, thereby allowing access to the patient from both sides of the chair apparatus 10. As shown, the backrest 82 extends upwardly from a rear end 36 of the seat portion 32 and is fully adjustable in increments so that the patient may be positioned anywhere from a vertical position to a reclined horizontal position. Each leg support 60 extends downwardly from a front end 34 of the seat portion 32, and the distal ends of the leg assemblies may be connected by a footrest 62, as shown in FIG. 1A. In some aspects, the leg supports 60 may be raised to a horizontal position so that the patient may be placed in a horizontal position, if desired.

Referring to FIGS. 1A-1B, the seat portion 32 of the chair apparatus 10 includes a body 41 that defines a recess 42. For example, the body 41 can have a front end 43 and a rear end 45. The front end 43 of the body 41 can define the recess 42 therein, recess extending rearwardly into the body 41 toward the rear end 45 of the body. The recess 42 is configured to slideably receive a portion of a generator assembly 17 of a corresponding imaging unit 14 therein when the chair apparatus 10 is positioned adjacent the imaging unit 14, such as when performing imaging (e.g., a mammography) or biopsy procedure on a patient. The imaging system 12 can be, for example, a breast imaging system, such as, for example, a 2D mammography system, 3D mammography system (also referred to as a tomosynthesis system), or a thermography system. The imaging system 12 can make use of one or more imaging modalities, such as, for example and without limitation, magnetic resonance imaging (MRI),/ultrasound, and/or X-ray imaging. An example imaging system 12 may include an imaging unit 14 rotatably coupled to a base 16. The imaging unit 14 can comprise an arm 18 that is rotatably coupled to the base 16. The arm 18 can have a generator assembly 17 at its first end and an image receptor plate 19 at its opposite second end. Typically, the arm 18 is disposed so that the generator assembly 17 is positioned above the image receptor plate 19. However, as previously noted, when performing “from below” procedures, the arm 18 of the imaging unit 14 is rotated 180° with respect to the base 18 of the imaging system 12 so that the generator assembly 17 is positioned below the image receptor plate 19, as shown in FIG. 1A. Note, embodiments of the disclosed chair apparatus 10 may be used to support a patient when capturing x-ray images, ultrasound images, magnetic resonance images, etc.

As best seen in FIGS. 3A and 3B, the surface or surfaces of the body 41 that define the recess 42 can have a profile that is U-shaped or substantially U-shaped. In some aspects, the body 41 of the seat portion 32 can comprise a cushion 52 and a frame 54 upon which the cushion rests. Optionally, in these aspects, the recess 42 in the seat portion 32 can be formed by both a recess in a front end of a portion of the cushion 52 of the seat portion 32 and a recess in the frame 54. For example, the cushion 52 can comprise a first portion 52a having a rear wall 44 and a pair of side walls 46 extending forwardly from opposite ends 44a,b of the rear wall. The rear wall rear wall 44 and the pair of side walls 46 can at least partly define the recess 42. As shown in FIG. 3B, the portion of the recess 42 formed in the frame 54 of the seat portion 32 can further be defined at least in part by a pair of side edges 56 and a rear edge 58 extending therebetween that extend inwardly beyond the sidewalls 46 and rear wall 44 formed by the first portion 52a of the cushion 52, thereby forming a shelf 47. In other aspects, the body 41 can be formed of a unitary structure. For example, optionally, in these aspects, the body 41 can be formed of a rigid structure (e.g., metal or polymer). Accordingly, in these optional aspects, the body 41 does not have a frame and a cushion that is separate from the frame. The size of the recess 42 formed in the body 41 of the seat portion 32 of various embodiments of the chair apparatus 10 may be differently sized depending on the size of the generator assembly 17 of the corresponding imaging unit with which the chair apparatus 10 will be used. For example, in some aspects, the recess 42 can have a depth of at least 4 inches, or at least 6 inches, or at least 8 inches, or from about 4 inches to about 12 inches. In various exemplary aspects, the recess 42 can have a width from about 6 inches to about 16 inches, or from about 8 inches to about 14 inches.

As shown, the cushion 52 of the seat portion 32 comprises the first portion 52a that at least partly defines the recess 42 that extends inwardly into the seat portion. The first portion 52a can be U-shaped or substantially U-shaped. As previously noted, the recess 42 facilitates positioning the disclosed chair apparatus 10 closer to an imaging unit 14 than is possible with known mammography chairs. Additionally, the cushion 52 of the seat portion 32 can also include a second portion 52b that is sized similarly to the recess 42 that is defined in the first portion 52a of the cushion 52. As such, when the chair apparatus 10 is not positioned adjacent the imaging unit 14, the second portion 52b of the cushion may be positioned in the recess defined in the front end of the first portion 52a of the cushion 52. As best seen in FIG. 3B, when the chair apparatus 10 (FIG. 1A) is in a sitting configuration for either ingress or egress of the patient, the second portion 52b of the cushion can be positioned in the recess 42 of the body 41 and positioned on the shelf 47 that is formed by the side edges 56 and rear edge 58 of the frame 54 of the body 41 of the seat portion 32. Once the patient is disposed on the seat portion 32, the attending physician or technician may remove the second portion 52b of the cushion 52 so that the chair apparatus 10 is in an imaging configuration, in which the chair apparatus may be positioned adjacent the imaging unit 14, as shown in FIG. 1B. As shown, a portion of the generator assembly 17 is slideably received within the recess 42 of the body 41 of the seat portion 32 of the chair apparatus 10 so that the patient may be positioned as close as possible to the generator assembly 17 and the image receptor plate 19.

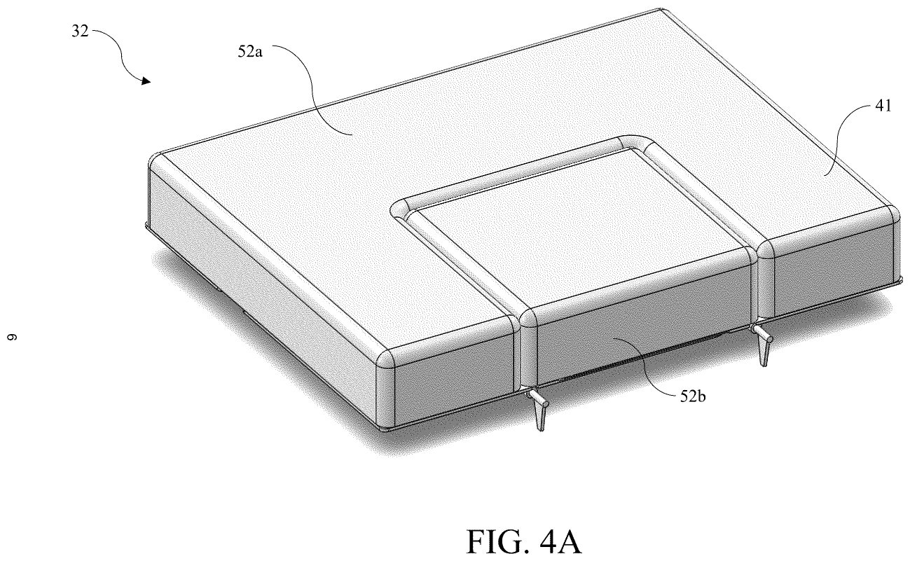

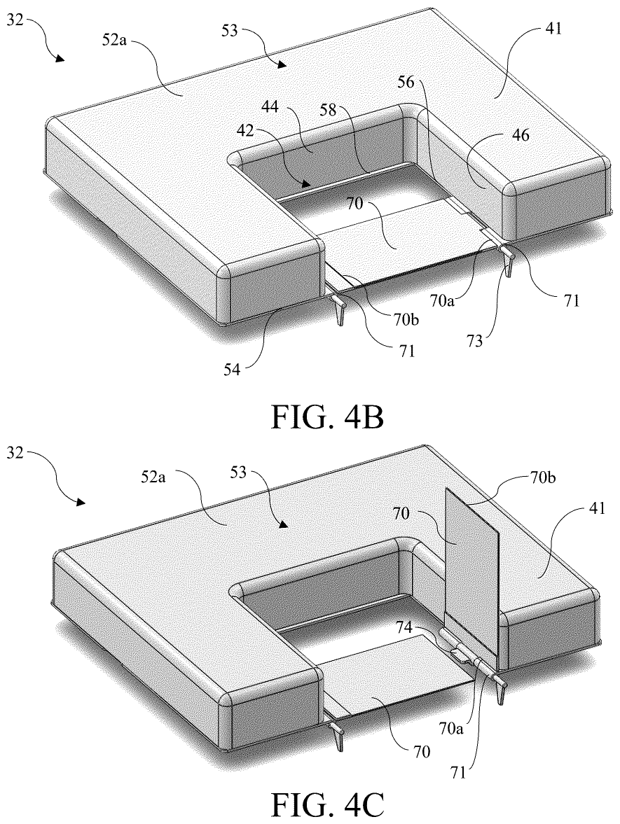

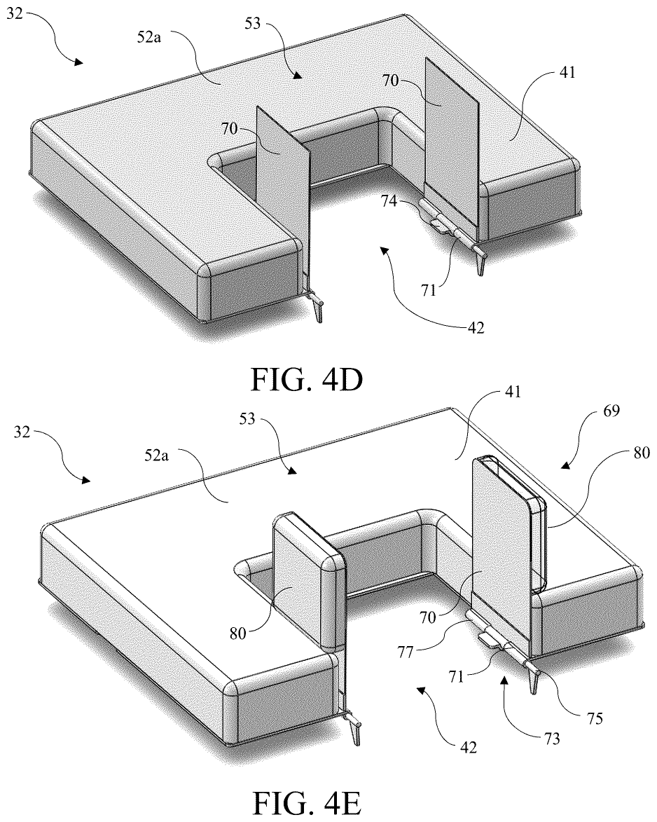

Referring now to FIGS. 4A-4E, in another embodiment of a seat portion 32 for use with the chair apparatus 10 in accordance with the present invention is shown. The seat assembly 32 can include a flap assembly 69 that prevents contact of the patient's legs with the generator assembly 17 of the corresponding imaging system 12. Referring additionally to FIG. 4A through 4E, the flap assembly 69 includes a pair of flaps 70, each flap being pivotably secured to a corresponding side edge 56 of the frame 54 of the seat assembly 30 that defines a portion of the recess 42. As best seen in FIG. 4C, each flap 70 includes a proximal end 70a that is pivotably secured to the corresponding side edge 56 of the frame 54 by a hinge 71, and a distal end 70b that extends above a top surface 53 of the first portion 52a of the cushion 52 when the flap 70 is disposed in the vertical position. In exemplary aspects, the flaps 70 can be rigid or substantially rigid to provide support to the legs of the patient that is sufficient to prevent contact between the legs of the patient and the generator assembly 17 of the imaging system 12 as further disclosed herein.

The height of each flap 70 from the proximal end 70a to the distal end 70b is less than the width of the recess 42 formed in the body 41 so that the flaps 70 may be nested together in a first horizontal position, as shown in FIG. 4B, when the chair apparatus 10 is not being actively utilized during a procedure. Each hinge 71 can include a horizontal tab 74 extending inwardly therefrom to support the corresponding flap 70 thereon when the flap 70 is placed in the horizontal position. When the flaps 70 are disposed in the horizontal position, a second portion 52b of the cushion 52 may be supported thereon in the sitting configuration, as shown in FIG. 4A, so that the cushion 52 has a continuous or generally continuous top surface to facilitate ingress and egress of the patient. Once a patient has been positioned on the seat assembly 30, the attending physician or technician may remove the second portion 52b of the cushion 52 from the recess 42 and rotate both flaps 70 to the vertical position, as shown in FIG. 4D. A lock feature 73 on each hinge 71 may be used to secure the corresponding flap 70 in the vertical position. The recess 42 of the body 41 seat portion 32 is now configured to slideably receive a portion of the generator assembly 17 of the corresponding imaging system 12 therein, as shown in FIG. 1A. Note, however, each flap 70 can also be provided with a corresponding pad 80 that may be removably secured to the portion of the flap 70 that extends above the top surface 53 of the first portion 52a of the cushion 52. The pads 80 are configured to provide comfort to the user as the user's legs do not come into contact with the metal surfaces of the flaps 70. Note, each hinge assembly 71 includes a horizontal tab 74 extending inwardly therefrom to support the corresponding flap 70 thereon when the flap is placed in the horizontal position.

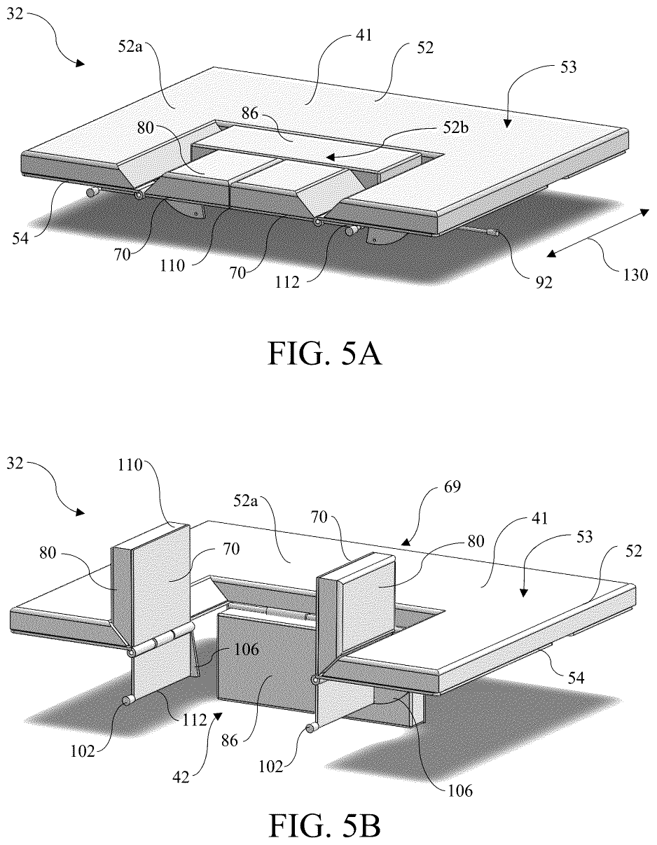

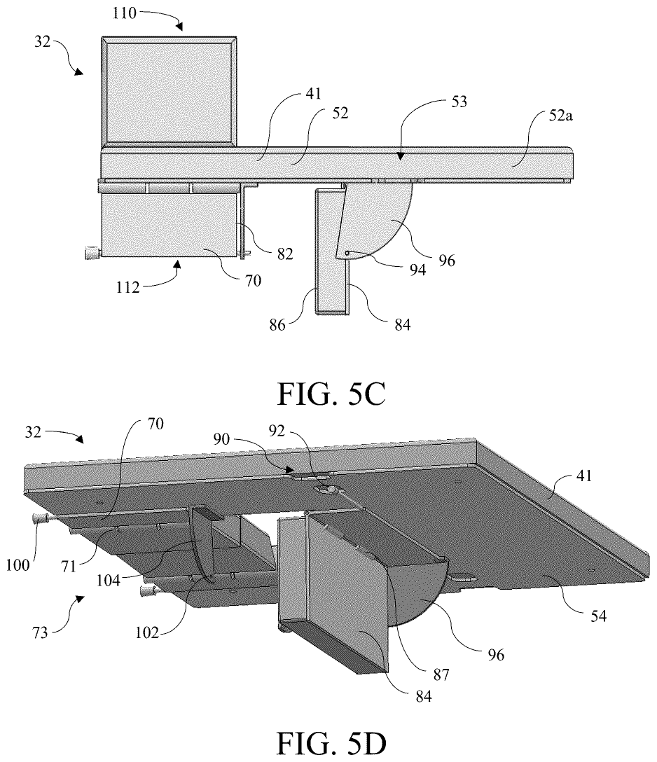

Referring to FIGS. 5A-5D, in some aspects, the pads 80 can serve as the second portion 52b of the cushion 52. For example, the pads 80 can extend upwardly from the flaps 70 to the same or substantially the same height as the top surface 53 of the first portion 52a of the cushion 52. For example, at least a portion of the upper surface of the pads 80 and at least a portion of the top surface 53 of the first portion 52a of the cushion 52 can be within ½ inch or within ¼ inch of each other along a vertical axis. In some aspects, the first portion 52a of the cushion 52 and the pads 80 can be shaped to permit the flaps to be pivoted without interference between the first portion of the cushion and the pads. For example, the first portion 54a and the pads 80 can have beveled edges to permit the flaps 70 to pivot while minimizing gaps between the pads 80 and the first portion 52a of the cushion 42. It is further contemplated that the first portion 52a of the cushion 52 and the pads 80 can be compressed against each other to permit additional movement of the flaps 70 once the first portion and the pads contact each other.

In some aspects, rear edges 82 of the flaps 70 can be spaced forwardly of a rear-most portion of the recess 42, such as, for example, the rear wall 44 formed by the first portion 52a of the cushion 52. This can permit more comfortable support for the user and accommodate more body shapes and sizes. Accordingly, in some aspects, the seat 32 can further comprise a third flap 84 that is pivotably coupled to the frame 54 and supporting a pad 86 rearward of the pads 80. For example, the third flap 84 can pivotably couple to the frame 54 at a hinge 87 positioned at the rear edge 58 of the frame 54. The pad 86 can cooperate with the pads 80 of the pair of flaps 70 to form the second portion 52b of the cushion 52. Accordingly, the third flap 84 can retain the pad 86 at the same or substantially the same height as the top surface 53 of the first portion 52a of the cushion 52 and the an upper surface of the pads 80.

A locking feature 90 can retain the third flap 84 in an elevated position. The locking feature 90 can be actuated to release the third flap 84 downwardly, thereby increasing the depth of the recess 42 to permit receipt of the generator assembly 17 (FIG. 1A) therein. The locking feature 90 can comprise a pin 92 (optionally, a spring-biased pin) that is coupled to the third flap 84 and radially spaced from the hinge 87. The pin 92 can be receivable into a retainer such as, for example, an opening 94 in a plate 96. Upon release of the pin 92 from the retainer, the third flap 84 can drop downwardly. In additional aspects, the retainer can comprise any hook, opening, slot, or protrusion that engages the pin 92 (or other suitable feature) for retaining the third flap 85 in the raised position. For example, the lock feature 73 of the hinge 71 can similarly be implemented for securing the third flap 84 in any position.

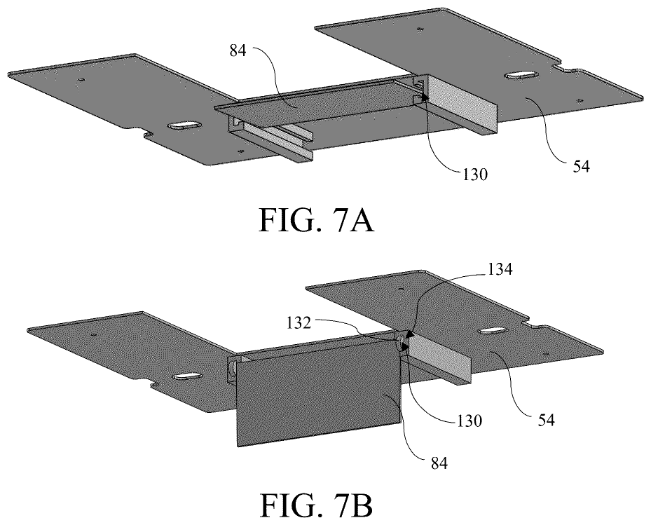

In additional aspects, and with reference to FIGS. 7A and 7B, the third flap 84 can be slidably received into a slot 130 of the frame 54 that supports a distal portion of the third flap in a cantilevered fashion, as shown in FIG. 7A. The third flap 84 can be withdrawn sufficiently from the slot 130 to permit the third flap 84 to pivot downwardly. Optionally, the third flap 84 can be coupled to at least one roller 132 for movement within the slot. The slot 130 can comprise a feature 134 (e.g., a protrusion, a wall, a narrowing, or an end cap) that inhibits the roller 132 from further forward movement.

As shown in FIGS. 5B-5D, in some aspects, the lock feature 73 can comprise a pin 100 that is coupled to the flap 70 and spaced radially from the hinge 71. The pin 100 can be receivable into a retainer such as, for example, an opening 102 in a plate 104. The lock feature 73 can secure each plate 70 in an upward configuration. In some aspects, the pin 100 can be spring-biased along the flap 70 toward a position that engages the opening 102 in the plate 104. In additional aspects, the retainer can comprise any hook, opening, slot, or protrusion that engages the pin 100 (or other suitable feature) for retaining the flap 70 in the raised position. For example, referring to FIG. 4E, the lock feature 73 can comprise a pin 75 that is threadedly received within a rear portion 77 of the hinge 71. The pin 75 can be rotated to tighten the pin within the rear portion 77 of the hinge 71 to inhibit movement of the hinge. The pin 75 can be loosened to permit the hinge to pivot.

In additional aspects, the flaps 70 and/or the third flap 84 can be actuated by an actuator. For example, the actuator can be a linear actuator (e.g., an electric actuator or a pneumatic or hydraulic piston) that is coupled to a flap by a linkage to impart rotational movement of the flap upon movement of the linear actuator. Optionally, each flap can comprise a respective actuator and linkage. In other aspects, a single actuator can be operably coupled to multiple flaps (e.g., both of the flaps 70 and, optionally, the third flap 84). In this way, the chair apparatus 10 can be moved about and between the seat configuration and the imaging configuration without a need for manual input.

Referring to FIGS. 5A-5D, in some aspects, the plate 70 can comprise a first end 110 and a second end 112. The second end 112 can bias against an underside of the frame 54 to support the first end 110 in a cantilevered fashion. Accordingly, in some aspects, the plates 70 need not lock in the lowered position (FIG. 5A).

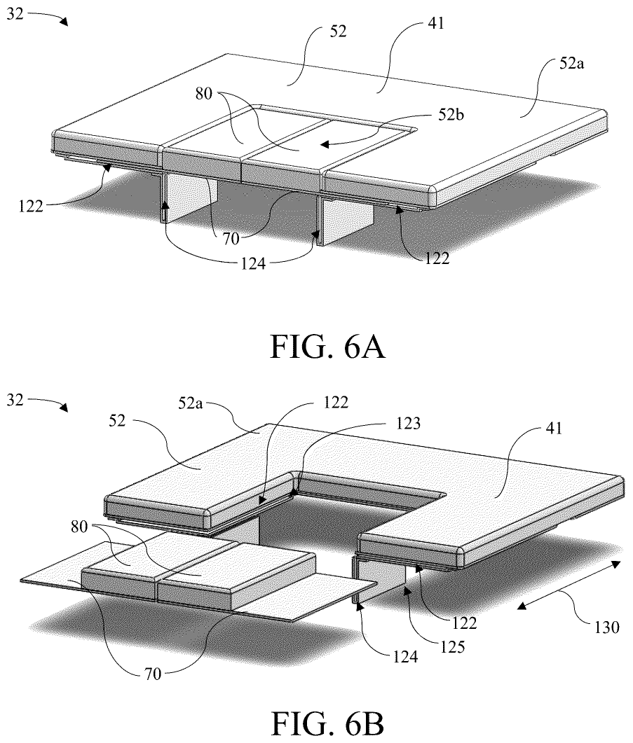

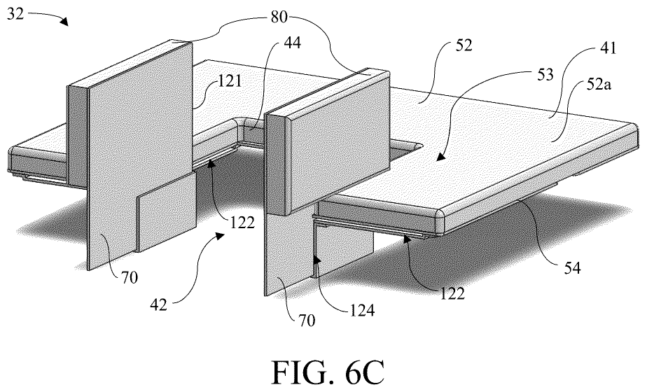

Referring to FIGS. 6A-6C, in additional aspects, the pads 80 can be supported in lowered and raised portions by one or more plates 120 that are slidably coupled to the frame 54. For example, the frame 54 can comprise a first pair of slots 122 that are configured to slidably receive a pair of plates 120 so that the pads 80 are generally horizontal. For example, the first pair of slots 122 can be generally horizontal. The frame 54 can further comprise a second pair of slots 124 that slidably receive the plates 120 so that the pads 80 extend upwardly above the top surface 53 of the first portion 52a of the cushion 52 and face outwardly to provide support to the legs of the patient that is sufficient to prevent contact between the legs of the patient and the generator assembly 17 of the imaging system 12.

In some aspects, the first pair of slots 122 can have an end 123 at a first position (e.g., a first depth) along a horizontal axis 130 extending from the front of the seat portion 32 to the rear that permits front edges of the pads 80 to be at or near a front edge of the first portion 52a of the cushion 52. In further aspects, rear edges of the pads 80 can be at or near the rear wall 44 formed by the first portion 52a of the cushion 52 when the plates 120 are received within the first pair of slots. The second pair of slots 124 can have an end 125 at a second position (e.g., a second depth) along the horizontal axis 130 that is different from the first position of the end of the first pair of slots so that at least a portion of the pads 80 are positioned forwardly of the front edge of the first portion 52a of the cushion 52. In this way, rear edges 121 of the plates 120 can be spaced forwardly to permit more comfortable support for the user and accommodate more body shapes and sizes.

In some aspects, a seat portion 32 can be provided for forming a seat assembly 30 of a chair assembly 10. For example, in some aspects, the seat portion 32 can be coupled directly to the vertical lift assembly 50. In other aspects, the seat portion 32 can be attached to a frame of the chair assembly 10 that, in turn, couples to the base assembly 20. The seat portion 32 can be attached to a remainder of the chair assembly 10 by one or more fasteners (e.g., screws, bolts, pins, or hooks). In further aspects, the seat portion 32 can be attached to the remainder of the chair assembly 10 by corresponding engaging surfaces that engage to inhibit relative movement therebetween. Such corresponding engaging surfaces can comprise, for example, one or more protrusions and corresponding slots. Optionally, the seat portion 32 can be adapted for an existing chair. For example, a conventional chair can have an existing seat portion removed, and the seat portion 32 can be provided in place of the removed seat portion. In other aspects, the chair assembly 10 can be designed for use with the disclosed seat portion 32.

### Exemplary Aspects

In view of the described apparatuses, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A seat portion for a chair apparatus for supporting a patient adjacent an imaging unit including a generator assembly and an image receptor plate, wherein the seat portion comprises:
a body having a front end and a rear end,
wherein the front end of the body of the seat portion defines a recess therein, the recess extending rearwardly into the body toward the rear end of the body, and
wherein the recess defined by the front end of the body is configured to slidably receive a portion of the generator assembly therein.

Aspect 2: The seat portion of aspect 1, wherein the body of the seat portion comprises a frame and a cushion supported thereon.

Aspect 3: The seat portion of aspect 2, wherein the cushion comprises a first portion, wherein the first portion of the cushion comprises a rear wall and a pair of side walls extending from opposite ends of the rear wall, wherein the recess is at least partly defined by the rear wall and the pair of side walls.

Aspect 4: The seat portion of aspect 2 or aspect 3, wherein the frame comprises at least one flap, wherein each flap of the at least one flap is pivotably mounted to the frame, wherein each flap of the at least one flap is pivotable between a horizontal position and a vertical position.

Aspect 5: The seat portion of aspect 4, wherein the at least one flap comprises a pair of rigid flaps, each rigid flap being configured to be disposed between a corresponding thigh of the patient and the generator assembly of the imaging unit when the portion of the generator assembly is slidably received in the recess of the seat portion.

Aspect 6: The seat portion of aspect 4 or aspect 5, wherein the cushion includes a first portion in which the recess is defined, and a second portion that is receivable within the recess, and wherein the at least one flap is configured to support the second portion of the cushion thereon when the at least one flap is in the horizontal position.

Aspect 7: The seat portion of aspect 6, wherein the at least one flap comprises a first flap pivotably mounted to the frame, and a second flap pivotably mounted to the frame.

Aspect 8: The seat portion of aspect 7, wherein the first flap and the second flap each include a distal end that extends above a top surface of the cushion when the first flap and the second flap are in the vertical position.

Aspect 9: The seat portion of aspect 7, wherein the first flap and the second flap each include a distal end that is disposed below a top surface of the cushion when the first flap and the second flap are in the vertical position.

Aspect 10: The seat portion of any one of aspects 5-9, wherein the cushion comprises a first portion having a rear wall, wherein the at least one flap further comprises a third flap pivotable about and between a lowered configuration and a raised configuration, wherein, in the raised configuration, the third flap is between the rear wall and the pair of flaps along a front-to-rear axis.

Aspect 11: The seat portion of any one of aspects 2-3, wherein the cushion comprises a first portion and at least one pad, wherein the seat assembly further comprises at least one plate, wherein each pad of the at least one pad is coupled to a respective plate of the at least one plate, wherein the frame defines at least one slot, wherein each slot of the at least one slot is configured to receive a respective plate of the at least one plate.

Aspect 12: The seat portion of aspect 11, wherein the at least one pad comprises a pair of pads, wherein the at least one plate comprises a pair of plates, wherein the at least one slot comprises a first pair of slots.

Aspect 13: The seat portion of aspect 12, wherein the frame further defines a second pair of slots, wherein each slot of the second pair of slots is configured to receive a respective plate of the pair of plates so that the pads extend upwardly above a top surface of the first portion of the cushion and outwardly from the plates away from each other.

Aspect 14: A chair apparatus comprising:
a base assembly configured to allow the chair apparatus to be moved along a support surface disposed beneath the chair apparatus; and
a seat assembly supported by the base assembly, wherein the seat assembly comprises the seat portion as in claim 1.

Aspect 15: A method of performing a breast imaging or biopsy procedure on a patient, the method comprising the steps of:
seating the patient on the seat assembly as recited in aspect 14 so that each leg of the patient is disposed on a corresponding side of the recess defined in the front end of the body of the seat portion; and
moving the chair apparatus toward the imaging unit so that a portion of the generator assembly is disposed within the recess defined in the front end of the body of the seat portion.

Aspect 16: The method of aspect 15, further comprising the step of:
capturing a radiological image of a portion of the patient.

Aspect 17: The method of aspect 15, further comprising the step of:
capturing one of an x-ray image, an ultrasound image, a thermographic image and a magnetic resonance imaging image of a portion of the patient.
Aspect 18: An imaging system for performing a breast exam on a patient, comprising:
an imaging unit including a generator assembly and an image receptor plate; and
a chair apparatus comprising:
   a base assembly configured to allow the chair apparatus to be moved along a support surface disposed beneath the chair apparatus; and
   a seat assembly supported by the base assembly, the seat assembly including a seat portion comprising a body having a front end and a rear end,
wherein the front end of the body of the seat portion defines a recess therein, the recess extending rearwardly into the body of the seat portion toward the rear end of the body of the seat portion, and
wherein the recess defined by the front end of the body is configured to slidably receive a portion of the generator assembly therein.

Aspect 19: The imaging system of aspect 18, wherein the body of the seat portion comprises:
a frame; and
a seat cushion supported thereon, wherein the seat cushion comprises a first portion and a second portion, wherein the first portion of the seat cushion at least partly defines the recess,
wherein the second portion is movable about and between a first configuration in which the second portion is within the recess and a second configuration in which the second portion is moved away from the recess to permit receipt of portion of the generator assembly therein.

Aspect 20: The imaging system of aspect 19, wherein the second portion comprises a plurality of pads.

Although the foregoing invention has been described in some detail by way of illustration and example for purposes of clarity of understanding, certain changes and modifications may be practiced within the scope of the appended claims.

What is claimed is:

1. A seat portion for a chair apparatus for supporting a patient adjacent an imaging unit including a generator assembly and an image receptor plate, wherein the seat portion comprises:
a body having a front end and a rear end,
wherein the front end of the body of the seat portion defines a recess therein, the recess extending rearwardly into the body toward the rear end of the body, and
wherein the recess defined by the front end of the body is configured to slidably receive a portion of the generator assembly therein.

2. The seat portion of claim 1, wherein the body of the seat portion comprises a frame and a cushion supported thereon.

3. The seat portion of claim 2, wherein the cushion comprises a first portion, wherein the first portion of the cushion comprises a rear wall and a pair of side walls extending from opposite ends of the rear wall, wherein the recess is at least partly defined by the rear wall and the pair of side walls.

4. The seat portion of claim 2, wherein the frame comprises at least one flap, wherein each flap of the at least one flap is pivotably mounted to the frame, wherein each flap of the at least one flap is pivotable between a horizontal position and a vertical position.

5. The seat portion of claim 4, wherein the at least one flap comprises a pair of rigid flaps, each rigid flap being configured to be disposed between a corresponding thigh of the patient and the generator assembly of the imaging unit when the portion of the generator assembly is slidably received in the recess of the seat portion.

6. The seat portion of claim 4, wherein the cushion includes a first portion in which the recess is defined, and a second portion that is receivable within the recess, and wherein the at least one flap is configured to support the second portion of the cushion thereon when the at least one flap is in the horizontal position.

7. The seat portion of claim 6, wherein the at least one flap comprises a first flap pivotably mounted to the frame, and a second flap pivotably mounted to the frame.

8. The seat portion of claim 7, wherein the first flap and the second flap each include a distal end that extends above a top surface of the cushion when the first flap and the second flap are in the vertical position.

9. The seat portion of claim 7, wherein the first flap and the second flap each include a distal end that is disposed below a top surface of the cushion when the first flap and the second flap are in the vertical position.

10. The seat portion of claim 5, wherein the cushion comprises a first portion having a rear wall, wherein the at least one flap further comprises a third flap pivotable about and between a lowered configuration and a raised configuration, wherein, in the raised configuration, the third flap is between the rear wall and the pair of flaps along a front-to-rear axis.

11. The seat portion of claim 2, wherein the cushion comprises a first portion and at least one pad, wherein the seat assembly further comprises at least one plate, wherein each pad of the at least one pad is coupled to a respective plate of the at least one plate, wherein the frame defines at least one slot, wherein each slot of the at least one slot is configured to receive a respective plate of the at least one plate.

12. The seat portion of claim 11, wherein the at least one pad comprises a pair of pads, wherein the at least one plate comprises a pair of plates, wherein the at least one slot comprises a first pair of slots.

13. The seat portion of claim 12, wherein the frame further defines a second pair of slots, wherein each slot of the second pair of slots is configured to receive a respective plate of the pair of plates so that the pads extend upwardly above a top surface of the first portion of the cushion and outwardly from the plates away from each other.

14. A chair apparatus comprising:
a base assembly configured to allow the chair apparatus to be moved along a support surface disposed beneath the chair apparatus; and
a seat assembly supported by the base assembly, wherein the seat assembly comprises the seat portion as in claim 1.

15. A method of performing a breast imaging or biopsy procedure on a patient, the method comprising the steps of:
seating the patient on the seat assembly as recited in claim 14 so that each leg of the patient is disposed on a corresponding side of the recess defined in the front end of the body of the seat portion; and
moving the chair apparatus toward the imaging unit so that a portion of the generator assembly is disposed within the recess defined in the front end of the body of the seat portion.

16. The method of claim 15, further comprising the step of:
capturing a radiological image of a portion of the patient.

17. The method of claim 15, further comprising the step of:
capturing one of an x-ray image, an ultrasound image, a thermographic image and a magnetic resonance imaging image of a portion of the patient.

18. An imaging system for performing a breast exam on a patient, comprising:
an imaging unit including a generator assembly and an image receptor plate; and
a chair apparatus comprising:
a base assembly configured to allow the chair apparatus to be moved along a support surface disposed beneath the chair apparatus; and
a seat assembly supported by the base assembly, the seat assembly including a seat portion comprising a body having a front end and a rear end,
wherein the front end of the body of the seat portion defines a recess therein, the recess extending rearwardly into the body of the seat portion toward the rear end of the body of the seat portion, and
wherein the recess defined by the front end of the body is configured to slidably receive a portion of the generator assembly therein.

19. The imaging system of claim 18, wherein the body of the seat portion comprises:
a frame; and
a seat cushion supported thereon, wherein the seat cushion comprises a first portion and a second portion, wherein the first portion of the seat cushion at least partly defines the recess,
wherein the second portion is movable about and between a first configuration in which the second portion is within the recess and a second configuration in which the second portion is moved away from the recess to permit receipt of portion of the generator assembly therein.

20. The imaging system of claim 19, wherein the second portion comprises a plurality of pads.
